(19) United States
(12) Patent Application Publication
(10) Pub. No.: US 2018/0271474 A1
(43) Pub. Date: Sep. 27, 2018

(54) MEDICAL IMAGING DEVICE ANALYZING SYSTEM

(71) Applicant: KONICA MINOLTA, INC., Tokyo (JP)

(72) Inventor: Yuuichi Nishijima, Tokyo (JP)

(73) Assignee: KONICA MINOLTA, INC., Tokyo (JP)

(21) Appl. No.: 15/923,180

(22) Filed: Mar. 16, 2018

(30) Foreign Application Priority Data
Mar. 24, 2017 (JP) 2017-058880

(51) Int. Cl. A61B 6/00

(52) U.S. Cl. CPC A61B 6/586; A61B 6/582; A61B 6/52

(57) ABSTRACT

A medical imaging device analyzing system, including: a medical imaging device which is provided in a facility of a client; and an analyzing device which collects one or a combination of device identification setting information, device status information and client's operation data concerning the medical imaging device from the medical imaging device, analyzes a problem in operation concerning the medical imaging device of the client based on the device identification setting information, the device status information or the client's operation data which are collected, and outputs an analysis result to the medical imaging device.

### CROSS-REFERENCE TO RELATED APPLICATIONS

The entire disclosure of Japanese patent Application No. 2017-058880, filed on Mar. 24, 2017, is incorporated herein by reference in its entirety.

### BACKGROUND

### 1. Field of the Invention

The present invention relates to a medical imaging device analyzing system.

### 2. Description of the Related Art

There have been known a variety of medical imaging devices to take images for medical use, as exemplified by radiation image capturing devices (Flat Panel Detectors: FPDs), computed tomography (CT) scanners, magnetic resonance imaging (MRI) scanners, ultrasound devices and breast image capturing devices (mammography scanners). Medical imaging devices also encompass radiation generators that emit radiation to radiation image capturing devices, radiographic image reading apparatuses that read signals from stimulable phosphor plates in computed radiography (CR) cassettes and so forth.

Such medical imaging devices have conventionally accompanied service to inform clients of expected malfunctions or lives of the devices, and solutions in case of any problem. For example, Japanese Patent Application Laid Open Publication No. 2011-072417 describes a system targeted at a radiation image capturing device, in which a life of the device is estimated by gathering the status of device usage and obtaining the degree of deterioration from images. Also, Japanese Patent Application Laid Open Publication No. 2001-209724 describes a technology of providing reference to solve a problem by letting a client access a database of service facility and displaying a list of solutions in case of any trouble.

However, the system in Japanese Patent Application Laid Open Publication No. 2011-072417 is basically meant to be mounted in a radiation image capturing device or a console that controls a radiation image capturing device and so forth. In a medical facility of a client which has not only the radiation image capturing devices but also other medical imaging devices such as computed tomography scanners and magnetic resonance imaging scanners, the above system or such has to be mounted in each device. Therefore the manufacturers or service personnel cannot access all the information.

The system in Japanese Patent Application Laid Open Publication No. 2011-072417 allows a client to check a list of solutions only by accessing a database of service facility. The system displays a solution which the client selects from the list. It is troublesome and inconvenient for the client to deal with many processes in search of an appropriate solution.

In order to solve these problems, the service personnel from the manufacturers could frequently visit the facility of the client to give the client advice to optimize the operations of medical imaging devices. However, the service may be insufficient because of the limited frequency of visits of the service personnel, considering the distribution of medical imaging devices not only domestic but also worldwide.

There are also cases where the client does not exercise the optimal operations of medical imaging devices, and where the client does not update the software of medical imaging devices or deal with modifying the setting parameters to the optimum. In such cases, there may be problems such as a radiation overdose to the subject patient. The service personnel have to frequently visit the facility of the client to solve problems like that, but it is not necessarily easy because of the reasons described above.

### SUMMARY

The present invention has been made in view of such problems and has the object to provide a medical imaging device analyzing system, in which a client can easily and accurately optimize the operation of medical imaging device without visits of the service personnel to the facility of client, with the status, operation and so forth of the medical imaging device in the facility of client monitored.

To achieve at least one of the abovementioned objects, according to an aspect of the present invention, a medical imaging device analyzing system reflecting one aspect of the present invention includes a medical imaging device which is provided in a facility of a client; and an analyzing device which collects one or a combination of device identification setting information, device status information and client's operation data concerning the medical imaging device from the medical imaging device, analyzes a problem in operation concerning the medical imaging device of the client based on the device identification setting information, the device status information or the client's operation data which are collected, and outputs an analysis result to the medical imaging device.

### BRIEF DESCRIPTION OF THE DRAWINGS

The present invention will be more fully understood from the detailed description given hereinbelow and the appended drawings which are given by way of illustration only, and thus are not intended as a definition of the limits of the present invention, and wherein:

FIG. 1 is a perspective view showing an external appearance of a radiation image capturing device as an example of a medical imaging device;

FIG. 2 is an enlarged view of radiation-detecting elements etc. of the radiation image capturing device;

FIG. 3 is a schematic view showing an example of a layout of an X-ray room;

FIG. 4 is a schematic view showing an example of a layout in a mobile medical vehicle on which the radiation generator is mounted;

FIG. 5 is a block diagram showing an example of an embodiment of a medical imaging device analyzing system; and

FIG. 6 is a chart showing an example of problems and solutions for the client's operation.

### DETAILED DESCRIPTION OF EMBODIMENTS

Hereinafter, one or more embodiments of the medical imaging device analyzing system according to the present invention will be described with reference to the drawings. However, the scope of the invention is not limited to the disclosed embodiments.

The embodiments described hereinafter adopt a radiation image capturing device as a medical imaging device and devices relating to the flat panel detector. However, the present invention may be embodied by one or a combination of a computed tomography scanner, a magnetic resonance imaging scanner, an ultrasound device and any other medical imaging devices, and the medical imaging device according to the present invention is not limited to a radiation image capturing device and devices relating to the radiation image capturing device.

### [On a Radiation Image Capturing Device]

Before describing the medical imaging device analyzing system in view of the present embodiments, a radiation image capturing device and a radiation generator adopted as examples of medical imaging devices are described. First, a radiation image capturing device is explained hereinafter. Though the radiation image capturing device described hereinafter is cassette-type (portable), the embodiments are also applicable to mounted-type (stationary) which is attached to the support or the like so as to be vertically movable.

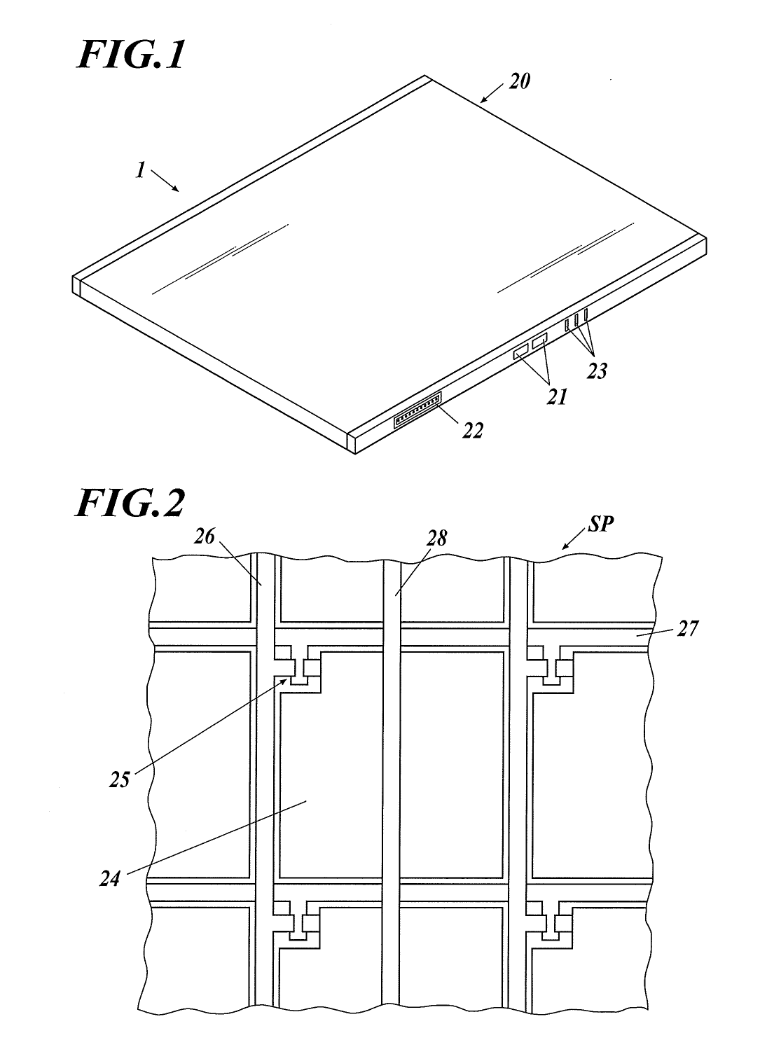

FIG. 1 is a perspective view showing an external appearance of a radiation image capturing device as an example of a medical imaging device, and FIG. 2 is an enlarged view of radiation-detecting elements etc. of the radiation image capturing device. The flat panel detector 1 is formed with a sensor panel SP with a sensor board not shown in the drawings and other parts stored in an enclosure 20 (in FIG. 1), wherein the sensor board has a plurality of radiation detection elements 24 (in FIG. 2, hereinafter also referred to as pixels) in a two dimensional (matrix) alignment on the surface.

As shown in FIG. 1, on the one side of the enclosure 20 of the radiation image capturing device 1, switches 21, a connector 22, indicators 23 and other parts are arranged. On the opposite side of the enclosure 20, there arranged is an antenna for wireless communications with external apparatuses, while not shown in the drawing.

As shown in FIG. 2, the radiation detecting elements 24 (in FIG. 2) have respectively a switch element 25 formed with a thin film transistor (TFT) and other components, and are connected to a signal line 26 through the switch element 25. The switch elements 25 are connected with scanning lines 27, and ON voltage or OFF voltage is applied to the switch elements 25 through the scanning lines 27 to set the switch elements 25 in ON or OFF state. Further, radiation detecting elements 24 are connected to bias lines 28 to apply reverse bias voltage.

The radiation image capturing device 1 also includes parts other than described above, for example, a scintillator that converts received radiation into electromagnetic waves of another wavelength such as visible light and irradiates the radiation detection elements 24, a readout IC that reads image data from the radiation detection elements 24, a memory unit that stores the image data to be read, a gate driver that switches voltage (ON voltage or OFF voltage) to be applied to scanning lines 27, a plurality of wireless modules that conduct wireless communications via an antenna, a built-in power supply that supplies necessary power to the readout IC, memory unit, gate driver, wireless modules and other functional parts in the device, and a control unit that controls the operation of each functional part.

### [On Devices for Capturing Images Using a Radiation Image Capturing Device]

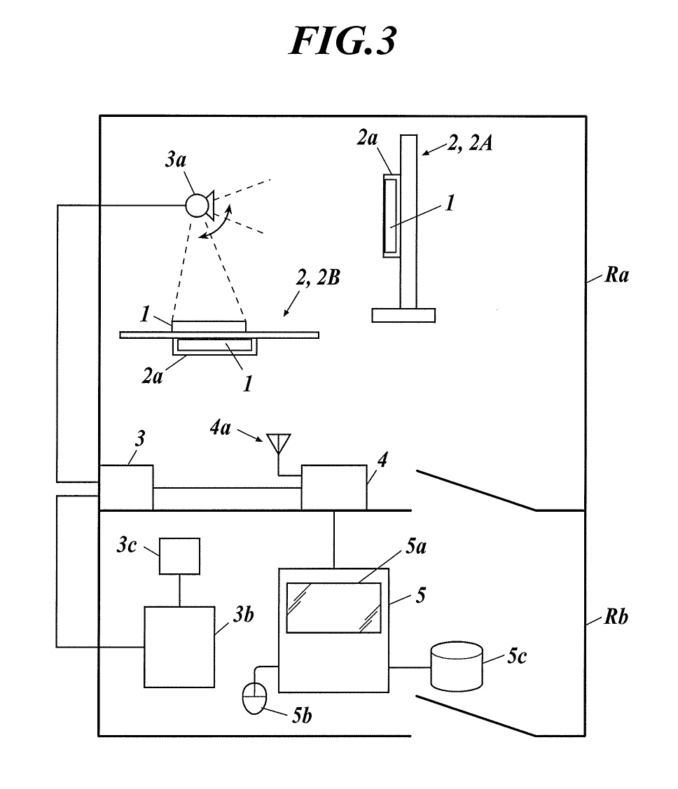

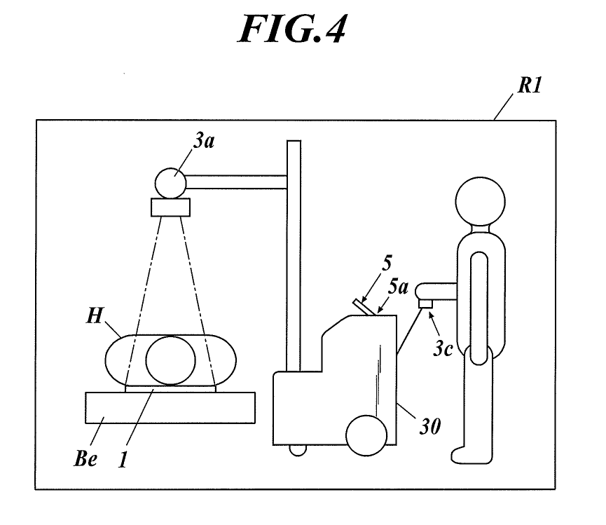

Next, some of the equipment concerning image capturing to be employed with the radiation image capturing device 1 is described. The radiation image capturing device 1 may be employed for image capturing in an X-ray room Ra as shown in FIG. 3, or in a sickroom R1 as shown in FIG. 4 whereto the radiation generator 3 and other devices are introduced by a mobile medical vehicle 30.

In a case where images are captured in an X-ray room Ra of a facility such as a hospital, as shown in FIG. 3, the radiation image capturing device 1 is mounted to a cassette holder 2a of a setting 2 (a wall stand 2A for upright positioning or a table 2B for dorsal positioning) and used for image capturing. The flat panel detector may also be inserted in a position between a top board of the table 2B for dorsal positioning and a subject in dorsal position on the top board not shown in the drawings.

The X-ray room Ra incorporates at least one radiation source 3a of a radiation generator 3 which irradiates a subject not shown in the drawings. When the tube voltage, tube current, radiation duration (or mAs value), etc. are set by an operator such as a radiographer, the radiation generator 3 controls the radiation source 3a to emit radiation of the dose according to the set tube voltage etc.

The X-ray room Ra also incorporates a router 4 combined with an access point 4a to interconnect the wired and wireless data communications among the devices inside and outside the X-ray room Ra. An anteroom (an operation room) Rb incorporates an operation console 3b of the radiation generator 3, and on the operation console 3b mounted is an irradiation switch 3c for a user to operate to command the initiation of irradiation to the radiation generator 3.

Installed in the anteroom Rb is a console 5 for controlling the radiation image capturing device 1, the radiation generator 3 and so forth to capture images. A display unit 5a comprising a cathode ray tube (CRT) and a liquid crystal display (LCD) and an input unit 5b are mounted to the console 5. And the console 5 is connected to or loaded with a memory unit 5c comprising a hard disk drive (HDD), etc. The console 5 is to store in the memory unit 5c the images captured and the information as to image capturing and image generation (for example, the conditions for image capturing and parameters used for image processing).

While omitted from FIG. 3, the console 5 is connected to the radiology information system, database (RIS), and so forth via the communication network or the like in the facility and transmits and stores the images obtained in accordance with the specifications decided by the facility and the information concerning image capturing and image generation in the required system or database. There are also cases where the console 5 and the radiation generator 3 are not connected to each other.

On the other hand, as described above and shown in FIG. 4, there may be a case where the radiation generator 3, console 5 and other devices are introduced to a sickroom R1 by the mobile medical vehicle 30 and images are captured therein. In such a case as this, the mobile medical vehicle 30 incorporates a radiation source 3a, a console 5 and other devices, and the radiation generator 3, router 4 and other devices are loaded inside the mobile medical vehicle 30, while not shown in the drawings.

In this case, the radiation image capturing device 1 is employed for image capturing by being inserted in a position between a bed Be and a patient as an object H or applied to the body of the patient. While not shown in the drawings, being connected to the communication network, the console 5 is configured to appropriately transmit and store the images and the information concerning image capturing and image generation in a system or database such as the radiology information system in the facility, after having completed image capturing.

### [On a Medical Imaging Device Analyzing System]

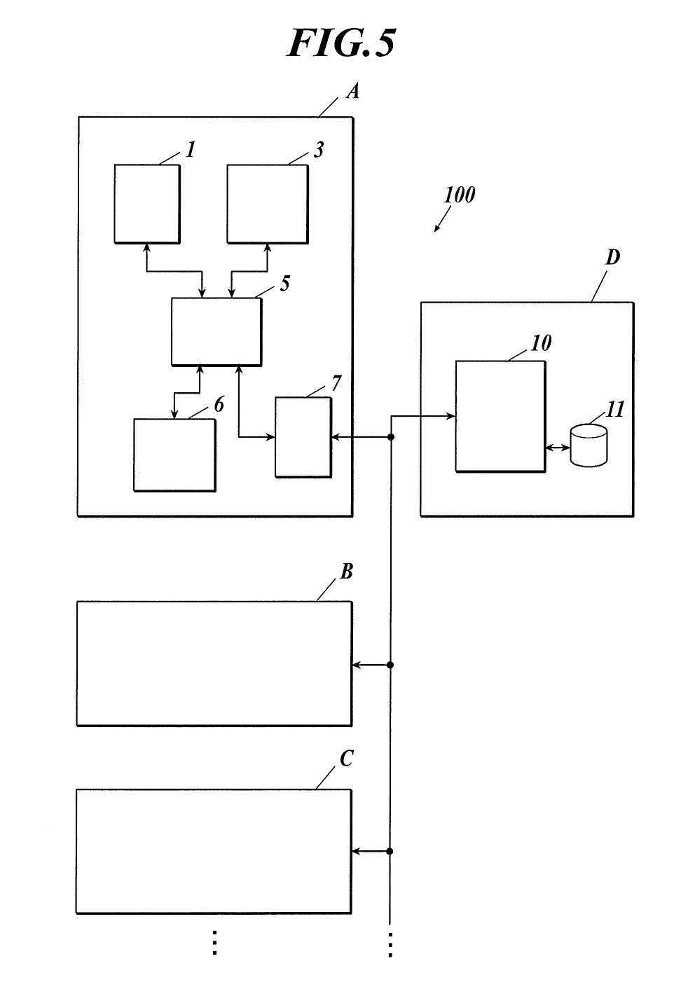

Next, a medical imaging device analyzing system 100 in accordance with the present embodiment is described hereinafter. In the embodiment, the medical imaging device analyzing system 100 includes facilities A, B, C and so forth such as a hospital or a clinic and a data center D as described in FIG. 5.

The facilities A, B, C, and so forth have a radiation image capturing device 1, a radiation generator 3, a console 5 (hereinafter referred to as medical imaging devices 1, 3, 5) and so forth. In FIG. 5 a component 6 describes a system or database such as the radiology information system, and is referred to as an internal system 6 hereinafter. The facilities A, B, C, and so forth have respectively a gateway unit 7 in the present embodiment.

The data center D has an analyzing device 10 which is connected to or loaded with a memory unit 11 consisting of a hard disk drive and other parts. In the present embodiment, the analyzing system 10 is to collect one or a combination of the data of device identification settings, device status and client's operating conditions of the medical imaging devices 1, 3, 5 from the facilities A, B, C, and so forth of the client.

The facilities A, B, C and so forth are the clients of the data center D. The number of the facilities which are the clients of the data center D may vary from 1, 2, to 4 or more. The data center D can be placed inside the facility if just one facility of a client belongs to the data center D. There is no need for the client to perform any operation when the analyzing device 10 of the data center D collects the information and data. First, the process where the analyzing device 10 of the data center D collects the information and data from the facility A is described hereinafter. The process where the analyzing device 10 of the data center D collects information and data from the facilities B, C, and so forth can be described likewise.

The data of the device identification settings concerning the medical imaging devices 1, 3, 5 in the facility (hereinafter referred to as the device identification setting information) is the information on the individual status of the medical imaging devices 1, 3, 5, for example, the sensitivity of the flat panel detector, the type of the scintillator of the flat panel detector, the size of the flat panel detector (sized 14×17 inches etc.), the types of pixel pitch/pixel binning of the flat panel detector, the type of the radiation detection elements (photo diode, charge coupled device etc.), the version information of the software, the information of the parameters of the device, the information of the operating system (OS) and so forth.

The device identification setting information does not vary while in operation. Thus, the device identification setting information is required to be stored in the memory 11 once by the analyzing system 10 of the data center D, only at the time of installation of the medical imaging devices 1, 3, 5 and in a case of modification for maintenance, for example, and not required to be collected thereafter.

The data of the device status concerning the medical imaging devices 1, 3, 5 in the facility (hereinafter referred to as the device status information) is the information variable while in the operation of the medical imaging devices 1, 3, 5, as exemplified by the wireless network information (reception level and forward rate/time) of the flat panel detector, the residual capacity of the built-in battery of the flat panel detector, the irradiation dose (including the irradiation dose received by the flat panel detector, the irradiation dose emitted by the radiation generator to the FPD and the exposure dose of the object patient), the hardware diagnostic information (of the flat panel detector, console and radiation generator), and the device operation log (of the flat panel detector and radiation generator).

The data of the client's operating conditions is the information on how the client employed the medical imaging devices 1, 3, 5, for example, the conditions for image capturing (tube voltage, tube current, irradiation duration (or mAs value), the size of the irradiation field, SID (distance between the irradiation source and the flat panel detector), usage of grid, etc.), the results of image capturing (radiographer information, patient information, region of body, medical imaging device employed, irradiation result, image captured, number of images successful and failed, etc.), the number of examinations and radiographies by each X-ray room (or mobile medical vehicle or operation room), the operation log (when and by whom the device is used), etc.

The device status information and the client's operation data are the information constantly updated. The analyzing system 10 of the data center D may collect the data from the facilities A, B, C, and so forth every time images are captured or regularly as configured. The collection measure is decided accordingly.

For example, there can be a configuration which allows the gateway unit 7 of the facility A to collect the information and data from the medical imaging devices 1, 3, 5, and the internal system 6 (i.e. the device or system where the necessary information and data mentioned above is stored) beforehand and then the analyzing device 10 of the data center D to access the gateway unit 7 of the facilities and collect the necessary information and data from the gateway unit 7.

There can be another configuration which allows the analyzing device 10 to collect from the gateway unit 7 the necessary information and data which the gateway unit 7 of the facility A has collected in accordance with the demand of the analyzing device 10 of the data center D. Also, there can be a configuration which allows the analyzing device 10 of the data center D to collect the necessary information and data directly from the medical imaging devices 1,3, 5, the internal system 6, and so forth in the facility A. In this case, the gateway unit 7 is not necessarily set up in the facility A.

After collecting, from the facility A of the client, one or a combination of the device identification setting information, the device status information, and the client's operation data as described above, the analyzing device 10 of the data center D analyzes the problems in the operation of the medical imaging devices 1, 3, 5 based on the device identification setting information, the device status information, and the client's operation data and feeds back the analysis results and feeds back the analysis results to the client.

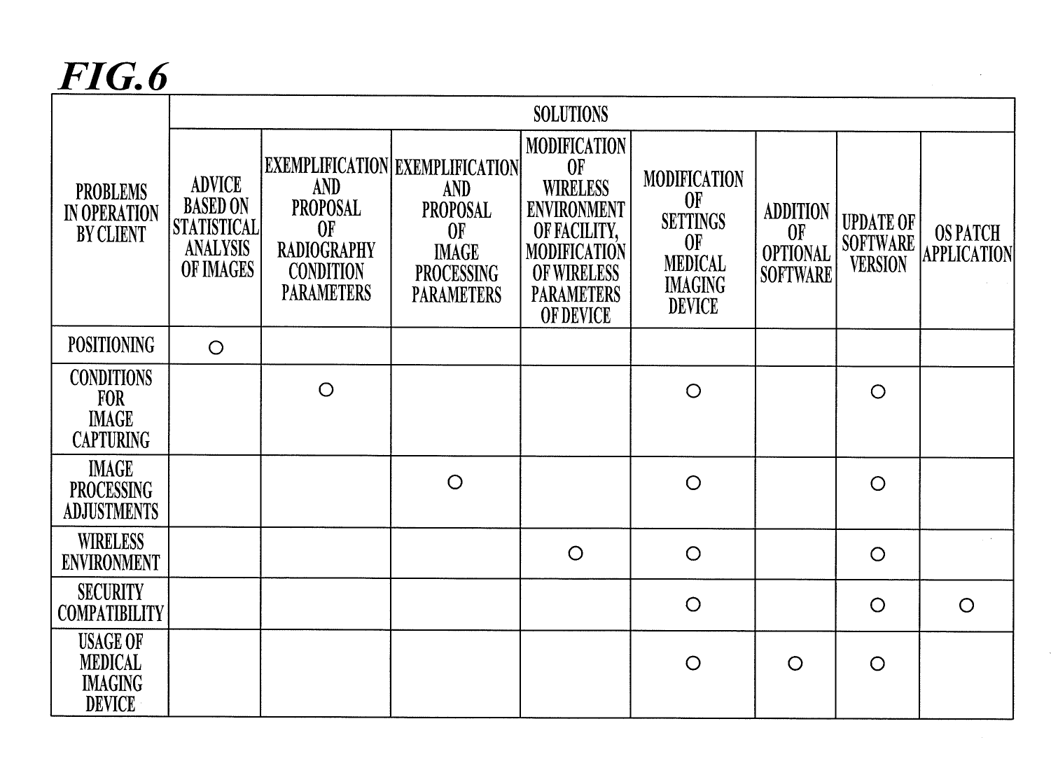

The problems in the client's operation can be exemplified by the position of the object in the image (hereinafter referred to as positioning), the conditions for image capturing (tube voltage, tube current, irradiation duration (or mAs value), size of the irradiation field, SID, usage of the grids, etc.), the image processing adjustment (adjusting method, parameters applied, etc.), the wireless network environment (channel used, access point used, spot of wireless communications, etc.), and the security compatibility (information of patch application of OS, encryption of wireless communications, setting of the screensaver, accessibility to the mobile media, etc.), the manner of usage of the medical imaging devices (screen operation, screen transition, operation of the flat panel detector, charging method of the flat panel detector, switching to the energy saving mode of the flat panel detector, method of the selection of the medical imaging devices to the object to be captured, calibration cycle, etc.), as shown in FIG. 6.

The solutions for the problems are, for example, advice based on the results of the statistical analysis, an exemplification and proposal of the parameters on image capturing and image processing, a modification of the facility wireless network environment, a modification of wireless network parameters of the device, a modification of the settings of the medical imaging device, an addition of the optional software, an update of the software version, an application of the OS patch, as shown in FIG. 6. The analyzing device 10 of the data center D is to feedback to the client the analysis results and the solutions for the problems in the operation.

To be concrete, the case where positioning is the problem in the client's operation is described hereinafter. The analyzing device 10 of the data center D extracts the images from the results of image capturing in the client's operation data among the information and data collected from the facility A. The images with proper positioning (for example, the region to be captured fits into the predetermined range of the image) are classified as “positioning OK,” and stored in the memory unit 11 (see FIG. 5) with the necessary information added. In this case, if nothing is wrong with positioning at least, it is not necessary to send feedback to the client.

However, if some of the images collected this time are not with proper positioning, for example, the analyzing system 10 of the data center D stores those images with improper positioning in the memory unit 11, classifying the images as “positioning NG” as reference. Accordingly the size of the flat panel detector used for image capturing with improper positioning is referred to from the device identification setting information in the facility which is stored in the memory unit 11.

And the analyzing device 10 of the data center D executes a statistical analysis of the images classified as “positioning OK” and as “positioning NG” among the images stored before in the memory unit 11 in which the same regions are captured with a larger sized radiation image capturing device 1 (medical imaging device 1) (for example, in what rate images are classified as “positioning NG,” etc.). When there is a high possibility that positioning would be more proper with a larger sized radiation image capturing device 1, the client receives advice to employ a larger sized panel to perform more proper image capturing (advice based on statistical analysis result of the image (see FIG. 6)).

The analyzing device 10 of the data center D may also identify the object patients of the images collected this time and classified as “positioning NG” from the patient information in the client's operation data collected simultaneously and extract the images of the same patients from the memory unit 11. There may be a case where the proper positioning have been attained in a sitting position (wherein the patient is sitting on a chair) while the image classified “positioning NG” this time is in a standing position. In that case, it is possible to feedback to the client advice that the patient be sitting while images are captured.

Further, the analyzing device 10 of the data center D may be configured to select the image with the most proper positioning from the images classified as “positioning OK” and stored in the memory unit 11, and output feedback showing the image as a sample of the proper positioning.

Another example of the problems in the client's operation is described hereinafter. If the analyzing device 10 of the data center D assesses that the radiation dose (the irradiation dose received by the flat panel detector, the irradiation dose emitted to the FPD by the radiation generator and the exposure dose of the object patient) in the device status information collected is far outside the regular range, the client may be provided with the condition parameters for image capturing (tube voltage, tube current, irradiation duration (or mAs value), source to image distance, etc.) of the images of other patients (or the patient concerned) in which the radiation dose of the object patients is within the predetermined range and the same region of body is properly captured, as shown in FIG. 6. The standard parameters of conditions for image capturing in capturing the region concerned may be exemplified or proposed.

As to image processing adjustment, the analyzing system 10 of the data center D may also be configured to feedback an exemplification or provide more proper image processing parameters and standard image processing parameters used for image processing adjustment of the region concerned.

On the other hand, the analyzing device 10 of the data center D may feedback to the client the analysis results and the solutions for the problems in the operation by offering the solutions as web content in the portal site for the facilities or by e-mail. In that case, the facility A has inside it a screen (ex. a screen 5a of the console 5, a screen of the administrative server not shown in the drawings) showing the analysis results and the solutions for the problems in the operation to inform a person in charge in the facility. The analyzing device 10 of the data center D may also be configured to offer the analysis results and the solutions for the problems in the operation printed on paper.

The configurations described above enable accurate notifications of the analysis results and the problems in the operation to the person in charge in the facility A of the client. Consequently the client can easily and accurately optimize the operation of the medical imaging devices 1,3, 5 without visits of the service personnel to the facilities concerned by the analyzing device 10 of the data center D monitoring the status and operation of the medical imaging devices 1, 3, 5 of the facility A of the client.

The facility A can apply the analysis results and the solutions for the problems in the operation, namely a modification of the settings, a modification of parameters, a software update, and so forth, to the medical imaging devices 1, 3, 5 with or without a check by the client, in addition to the methods described above where the modification of the settings, the modification of parameters, the update of software version and such like in the medical imaging device are displayed on the screen of the device in the facility.

The configurations described above can optimize the medical imaging devices 1, 3, 5, with the settings being automatically modified, the parameters being modified to be appropriate, or the software version being updated. Consequently the analyzing device 10 of the data center D can optimize the operation of the medical imaging devices 1,3, 5 without visits of the service personnel to the facilities concerned by monitoring the status and operation of the medical imaging devices 1, 3, 5 of the facility A of the client.

What is described above is a case where the analyzing device 10 of the data center D collects the information and data from the facility A of the client and feeds back the analysis results and the solutions for the problems in the operation to the facility A. As shown in FIG. 5, however, the analyzing device 10 of the data center D may be configured to collect the information and data (i.e. the device identification setting information, the device status information and the client's operation data), from more than one of the facilities A, B, C, and so forth.

In that case, when the analyzing device 10 of the data center D collects one or a combination of the device identification setting information, the device status information, and the client's operation data of the facilities A, B, C, and so forth of a plurality of the clients, and analyzes the problems in the operation of the medical imaging devices 1, 3, 5 by one client based on the information and data collected from the facility of the one client (for example, the facility A), the analyzing device 10 may use the analysis results of the problems in the operation of the medical imaging device 1, 3, 5 in the facilities of the other clients (for example, the facilities B, C, and so forth) for the above analysis and feedback the analysis results and the solutions for the problems in the operation to the one client.

In other words, there can be configurations to analyze not only the information and data collected from the facility A of the client but also the information and data collected from the other facilities B, C, and so forth and feedback the analysis results and the solutions for the problems in operation to the facility A.

The configurations enable the facility A of the client to easily and accurately optimize the medical imaging devices 1, 3, 5 without visits of the service personnel to the facility A by, for example, feeding back to the facility A advice to perform, for medical imaging devices 1, 3, 5, an operation performed in the facility B, when the status of the operation of the medical imaging devices 1, 3, 5 is better in the facility B than in the facility A and the analyzing device 10 of the data center D analyzes the cause for the better status and finds out that it is because of the operation of the facility B which is not applied by the facility A.

As described hereinbefore, the medical imaging device analyzing system 100 in the embodiment is configured to collect one or a combination of the device identification setting information, the device status information, and the client's operation data of the medical imaging devices 1, 3, 5 from the facilities A, B, C, and so forth of the client(s), analyze the problems in the operation concerning the medical imaging devices 1, 3, 5 based on the device identification setting information, the device status information, and/or the client's operation data and feedback the analysis results to the client concerned.

Therefore, the client can easily and accurately optimize the operation of the medical imaging devices 1, 3, 5 with the status and operation of the medical imaging devices 1, 3, 5 of the facilities A, B, C, etc. of the clients being monitored and without visits of the service personnel to the facilities concerned.

It is obvious that the present invention is not limited to the above-described embodiments and modifications and applications may be made without departing from the scope of the invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical imaging device analyzing system, comprising:
a medical imaging device which is provided in a facility of a client; and
an analyzing device which collects one or a combination of device identification setting information, device status information and client's operation data concerning the medical imaging device from the medical imaging device, analyzes a problem in operation concerning the medical imaging device of the client based on the device identification setting information, the device status information or the client's operation data which are collected, and outputs an analysis result to the medical imaging device.

2. The medical imaging device analyzing system according to claim 1, wherein the analyzing device outputs a solution for the problem in operation as the analysis result to the medical imaging device.

3. The medical imaging device analyzing system according to claim 1, wherein the medical imaging device includes a display which displays the analysis result output from the analyzing device.

4. The medical imaging device analyzing system according to claim 1, wherein the medical imaging device adopts the analysis result output from the analyzing device, with or without a check by the client.

5. The medical imaging device analyzing system according to claim 1, further comprising a plurality of medical imaging devices which are provided in a plurality of facilities of clients, wherein
the analyzing device collects one or a combination of device identification setting information, device status information and client's operation data concerning the plurality of medical imaging devices from the respective medical imaging devices,
when the analyzing device analyzes a problem in operation concerning one medical imaging device of a client based on device identification setting information, device status information or client's operation data collected from the one medical imaging device, the analyzing device analyzes the problem in operation by using an analysis result of a problem in operation concerning another medical imaging device, and
the analyzing device outputs an analysis result or a solution for the problem in operation to the one medical imaging device.
